(19) United States
(12) Patent Application Publication
(10) Pub. No.: US 2025/0277233 A1
(43) Pub. Date: Sep. 4, 2025

(54) EXOGENOUS GENE CONTROLLABLE EXPRESSION VIRUS PACKAGING VECTOR AND PACKAGING METHOD

(71) Applicant: OBIO TECHNOLOGY (SHANGHAI) CORP., LTD., Shanghai (CN)

(72) Inventors: Xinglin YANG, Shanghai (CN); Jiali YANG, Shanghai (CN); Guodong JIA, Shanghai (CN); Ruiju YANG, Shanghai (CN); Qingrui YOU, Shanghai (CN)

(21) Appl. No.: 18/265,857

(22) PCT Filed: Dec. 6, 2021
(86) PCT No.: PCT/CN2021/135817

§ 371 Date: Jun. 7, 2023

(30) Foreign Application Priority Data
Dec. 7, 2020 (CN) 202011418490.9
Dec. 7, 2020 (CN) 202011419528.4

(51) Int. Cl. C12N 15/86

(52) U.S. Cl. CPC C12N 15/86; C12N 2710/10043; C12N 2710/10052; C12N 2750/14143; C12N 2750/14152

(57) ABSTRACT

The present application relates to the technical field of biology, and specifically, to an exogenous gene controllable expression virus packaging vector and a packaging method. The virus packaging vector has two ITR fragments and a gene expression cassette inserted between the two ITR fragments. The gene expression cassette comprises a promoter, a repressor operator, and an optional target gene that are connected in sequence. When there is a repressor, the repressor operator can repress the expression of a downstream target gene thereof.

### CROSS-REFERENCE TO RELATED APPLICATIONS

This application claims priority of Chinese Patent Application No. 202011418490.9, filed with the Chinese Patent Office on Dec. 7, 2020, entitled “Adenovirus packaging method for exogenous gene controllable expression”, and Chinese Patent Application No. 202011419528.4, filed with the Chinese Patent Office on Dec. 7, 2020, entitled “Adeno-associated virus packaging method for exogenous gene controllable expression”. The contents of the above identified applications are hereby incorporated herein in their entireties by reference.

### TECHNICAL FIELD

The present disclosure relates to the field of biotechnology, in particular, to a vector for viral packaging with a controllable expression of exogenous genes and a method for packaging the same.

### BACKGROUND

Viral vectors can bring genetic materials into cells via the principle of molecular mechanism that viruses bind to cell surface receptors with a high affinity and transmit their genomes into certain types of cells for infection. Viral vectors are widely used in basic research, gene therapy or vaccines.

Currently, viruses commonly used as viral vectors include adeno-associated virus, adenovirus, and the like.

Adeno-associated virus (AAV), belonging to the Dependovirus genus of the Parvoviridae family, is a class of single-stranded DNA-deficient viruses with the simplest structure found so far. The replication of AAV requires the participation of a helper virus (usually an adenovirus). The AAV genome encodes cap and rep genes between two inverted terminal repeats (ITRs). ITRs play a decisive role in the replication and packaging of the virus. The cap gene encodes the viral capsid protein, and the rep gene is involved in the replication and integration of the virus. AAV is capable of infecting a variety of cells. In the presence of the product of rep gene, viral DNA readily integrates into human chromosome 19. AAV has the characteristics such as high safety, low immunogenicity, wide host range, and the capability of mediating long-term stable expression of exogenous genes in animals. AAV is one of the most promising vectors in the field of gene therapy. AAV plays an important role and has great demand in the fields of neuroscience research and gene therapy for diseases. In medical research, AAV is useful in the research of gene therapy for various diseases, including in vivo and in vitro experiments. At the same time, as a characteristic gene transfer vector, AAV is also widely used in gene function research, disease model construction, preparation of gene-knockout mice, and the like.

Adenovirus (ADV) is particularly suitable for use as a viral vector. Adenovirus is a non-enveloped virus with a diameter of approximately 90 nm to 100 nm, containing a nucleocapsid and a linear double-stranded DNA genome. Viral nucleocapsid includes penton capsids and hexon capsids. A unique fiber is associated with each penton matrix and facilitates the attachment of the virus to a host cell via the coxsackievirus-adenovirus receptor on the cell surface of the host cell. More than 50 serotype strains of adenovirus have been identified, most of which cause respiratory infections, conjunctivitis, and gastroenteritis in humans. The genome of adenovirus typically replicates as episomal components in the nucleus of the host cell rather than integrating into the host genome. The genome of adenovirus contains 4 early transcription units (i.e., E1, E2, E3 and E4), which mainly exert regulatory functions and make a preparation for viral replication in host cells. The genome also contains five late transcription units (i.e., L1, L2, L3, L4, and L5), which are transcribed into structural proteins including penton (L2), hexon (L3), scaffolding protein (L4) and fibrin (L5), where the structural proteins are under the control of a single promoter. Each end of the genome contains inverted terminal repeat (ITR) sequences necessary for viral replication.

Current studies of viral vectors focus on aspects of infectivity, specificity, packaging capacity or the like, while few attentions have been paid to effects of exogenous genes packaged (i.e., gene of interest, GOI) on the virus. In practice, a considerable proportion of exogenous genes result in not being able to obtain effective viral particles. The reasons include the follows. In the viral packaging process, an exogenous gene is usually driven by a strong promoter, and its expression has unpredictable effects on the condition of cells, the packaging, release, and other processes of viruses.

### SUMMARY

Various exemplary embodiments disclosed in the present disclosure provide a vector for viral packaging, including two inverted terminal repeats, and a gene expression cassette inserted between the two inverted terminal repeats. The gene expression cassette includes a promoter, a repressible operon and an optional gene of interest which are linked in sequence. The repressible operon is capable of repressing expression of the gene of interest downstream of the repressible operon in the presence of a repressor.

According to another aspect, the present disclosure relates to a viral vector packaging system, including the vector for viral packaging as described above and a backbone vector.

According to yet another aspect, the present disclosure relates to a method for packaging a virus, including transferring the viral vector packaging system as described above into a host cell, and performing packaging in the presence of a repressor.

Compared to conventional vectors for viral packaging, by using the vectors for viral packaging of the present disclosure, there is no need to determine whether the exogenous gene (i.e., the gene of interest) generates an effect on the virus. In the absence of a repressor, the repression system would have no function and thus no effect on the expression of the gene of interest. In the case where significantly lower viral release than the average level is observed, in cooperation with a helper plasmid capable of expressing a repressor protein, the process of viral packaging can be carried out with the expression of exogenous gene inhibited, thereby eliminating the influence of the exogenous gene and increasing the yield of specific virus, without reconstructing a new shuttle vector, and thus saving a lot of time and human and material resources.

### BRIEF DESCRIPTION OF THE DRAWINGS

In order to more clearly illustrate the technical solutions in the specific embodiments or conventional techniques of the present disclosure, the accompanying drawings needed to be used in the description of specific embodiments or conventional techniques are briefly described in the below: It is apparent that the accompanying drawings as described below show some embodiments of the present disclosure, and other drawings can also be obtained based on these drawings by those of ordinary skill in the art, without creative work.

FIGS. 1 to 5 show the plasmid maps of vectors pcDNA3.1-CymR, CMV-CuO, EF1a-CuO, SFH-CuO and CAG-CuO, respectively, in an embodiment of the present disclosure.

FIG. 6 shows the results of verification of repression efficiency of CymR-CuO in a lentiviral vector in an embodiment of the present disclosure.

FIG. 7 shows the plasmid map of CMV-TrpO in an embodiment of the present disclosure.

FIG. 8 shows the results of verification of repression efficiency of tryptophan operon system in a lentiviral vector in an embodiment of the present disclosure.

FIG. 9 shows the plasmid map of EF1a-ToxO in an embodiment of the present disclosure.

FIG. 10 shows the results of verification of repression efficiency of diphtheria toxin repressor regulatory system in a lentiviral vector in an embodiment of the present disclosure.

FIG. 11 shows the plasmid map of SFH-LacO in an embodiment of the present disclosure.

FIG. 12 shows the result of verification of repression efficiency of lactose operon system in a lentiviral vector in an embodiment of the present disclosure.

FIG. 13 is a schematic diagram showing insertion sites of TrpO element relative to TATA Box of CMV in an embodiment of the present disclosure.

FIG. 14 shows the influence of different insertion sites of TrpO element on repression efficiency in an embodiment of the present disclosure.

FIG. 15 is a schematic diagram showing insertion sites of CuO element relative to TATA Box of CMV in an embodiment of the present disclosure.

FIG. 16 shows the influence of different insertion sites of CuO element on repression efficiency in an embodiment of the present disclosure.

FIG. 17 is images showing the results for fluorescence observation under expression of NLS-iCre gene by an adenovirus vector in an embodiment of the present disclosure.

FIG. 18 shows the results of titer test under expression of NLS-iCre gene by an adenovirus vector in an embodiment of the present disclosure.

FIGS. 19 to 21 respectively show plasmid maps of pShuttle-CMV-iCre-3×Flag-P2A-sfGFP, pShuttle-CMV-TrpO-iCre-3×Flag-P2A-sfGFP, and pShuttle-CMV-CuO-iCre-3×Flag-P2A-sfGFP in an embodiment of the present disclosure.

FIG. 22 is images showing the results for fluorescence observation under expression of AceD81S gene by an adenovirus vector in an embodiment of the present disclosure.

FIG. 23 shows the results of titer test under expression of AceD81S gene by an adenovirus vector in an embodiment of the present disclosure.

FIGS. 24 to 26 show plasmid maps of pShuttle-CMV-AceD81S-mOrange2, pShuttle-CMV-TrpO-AceD81S-mOrange2, and pShuttle-CMV-CuO-AceD81S-mOrange2, respectively in an embodiment of the present disclosure.

FIG. 27 shows schematic diagrams of principles of A) adenovirus packaging in the absence of repressible operon, B) adenovirus packaging in the presence of Cumate-CuO regulable system, and C) adenovirus packaging in the presence of tryptophan operon.

FIG. 28 shows the results of virus titer test under expression of GPR78 gene by an adeno-associated virus vector in an embodiment of the present disclosure.

FIGS. 29 to 31 show plasmid maps of vectors pAAV-CMV-GPR78-3×FLAG-WPRE, pAAV-CMV-CuO-GPR78-3×FLAG-WPRE, and pAAV-CMV-TrpO-GPR78-3×FLAG-WPRE, respectively, in an embodiment of the present disclosure.

FIG. 32 shows the results of virus titer test under expression of Cdkn1a gene by an adeno-associated virus vector in an embodiment of the present disclosure.

FIGS. 33 to 35 show plasmid maps of vectors pAAV-CMV-Cdkn1a-3×FLAG-WPRE, pAAV-CMV-CuO-Cdkn1a-3×FLAG-WPRE, and pAAV-CMV-TrpO-Cdkn1a-3×FLAG-WPRE, respectively, in an embodiment of the present disclosure.

FIG. 36 shows schematic diagrams of principles of A) adeno-associated virus packaging in the absence of repressible operon, B) adeno-associated virus packaging in the presence of tryptophan operon, and C) adeno-associated virus packaging in the presence of Cumate-CuO regulable system.

### DETAILED DESCRIPTION

Reference to embodiments of the present disclosure will be made in detail and one or more examples of which are described below. Each of the examples is provided by way of explanation and does not limit the present disclosure. In fact, it will be apparent to those skilled in the art that various modifications and variations can be made to the present disclosure without departing from the scope or spirit of the present disclosure. For example, features illustrated or described as a part of one embodiment is applicable to another embodiment to generate a still further embodiment.

Thus, it is intended that the present disclosure covers such modifications and variations falling within the scope of the appended claims and their equivalents. Other subjects, features and aspects of the present disclosure are disclosed in or are apparent from the following detailed description. It is understood by those skilled in the art that the present discussion is a description of exemplary embodiments only and is not intended to limit broader aspects of the present disclosure.

Unless otherwise defined, all technical and scientific terms used herein have the same meaning as commonly understood by those skilled in the technical field to which this present disclosure belongs. The terms used herein in the specification of the present disclosure are only for the purpose of describing specific embodiments and are not intended to limit the present disclosure. As used herein, the term “and/or” includes any or all combinations of one or more of the associated listed items.

The present disclosure relates to a vector for viral packaging, including two inverted terminal repeats, and a gene expression cassette inserted between the two inverted terminal repeats. The gene expression cassette includes a promoter, a repressible operon and an optional gene of interest which are linked in sequence. The repressible operon is capable of repressing expression of the gene of interest downstream of the repressible operon in the presence of a repressor.

In the present disclosure, the expression of the gene of interest can be regulated through the repressible operon, thereby achieving the selective inhibition of the expression of exogenous gene in the cells for adenovirus packaging, avoiding the negative influence of the exogenous gene on the packaging and production of adenovirus, and improving the production efficiency and titer of virus. Meanwhile the expression level of the exogenous gene in a target cell is not reduced, which will not affect the therapeutic effect or study of the function of exogenous gene.

Term “vector” in the present disclosure refers to a nucleic acid delivery vehicle into which a polynucleotide can be packaged. In the case where a vector enables the expression of the protein encoded by the inserted polynucleotide, the vector is called an expression vector. A vector can be introduced into a host cell by transformation, transduction, or transfection, so that the genetic material element carried by the vector can be expressed in the host cell. In an embodiment, the vector is, but not limited to, a plasmid.

In some embodiments, the vector for viral packaging is a vector for adenovirus packaging or a vector for adeno-associated virus packaging.

In some embodiments, the repressible operon is selected from a tryptophan operon and/or a Cumate-CuO regulable system.

In some embodiments, the repressible operator has one or more copies.

In some embodiments, a distance between a site where a TrpO element of a tryptophan operon is inserted into the gene expression cassette and a TATA BOX of the promoter is less than or equal to 18 nucleotides, such as 17, 16, 15, 14, 13, 12, 11, 10, 9, 8, 7, 6, 5, 4, 3, 2 or 1 nucleotide(s).

In some embodiments, a distance between a site where a CuO element of a Cumate-CuO regulable system is inserted into the gene expression cassette and a TATA BOX of the promoter is 40 to 50 nucleotides, such as 41, 42, 43, 44, 45, 46, 47, 48 or 49 nucleotides.

The distance between the site where the above-mentioned element of the repressible operator is inserted and the TATA BOX of promoter refers to the number of nucleotide(s) between a first nucleotide (exclusive) at the 5′ end of the element and a first nucleotide (exclusive) at the 3′ end of the TATA BOX.

It should be noted that it is generally considered that the eight nucleotides (including the 8th nucleotide) following the TATA BOX are considered to belong to a core region of the promoter. In an embodiment, the distance between the TrpO element and the TATA BOX of the promoter is greater than 8 nucleotides. In the case where the TrpO element has a sequence that is overlapped with said eight nucleotides, the TrpO element can be closer to the TATA BOX.

The “promoter” is a DNA sequence that directs the binding of RNA polymerase and thereby initiates RNA synthesis. As used in the present disclosure, the promoter allows expression in a wide variety of types of cells and tissues. The promoter may be a non-cell-specific promoter. Alternatively; the promoter may be a cell-specific promoter, a cell type-specific promoter, a cell lineage-specific promoter, or a tissue-specific promoter, which allows the expression in their respective species-restricted types of cells or tissues. In particular embodiments, it may be desirable to use expression control sequences specific to cells, cell types, cell lineages or tissues, to achieve the cell type-specific, cell lineage-specific or tissue-specific expression of a desired polynucleotide sequence, e.g., expression of a nucleic acid encoding a polypeptide only in a subgroup of cell types, cell lineages, or tissues, or at specific developmental stages.

Exemplary examples of tissue-specific promoters include but are not limited to, B29 promoter (expressed in B cells), runt transcription factor (CBFa2) promoter (specifically expressed in stem cells), CD14 promoter (expressed in monocytes), CD43 promoter (expressed in leukocytes and platelets), CD45 promoter (expressed in hematopoietic cells), CD68 promoter (expressed in macrophages), CYP4503A4 or ALB promoter (expressed in hepatocytes), desmin promoter (expressed in muscle cells), elastase 1 promoter (expressed in pancreatic acinar cells), endothelial glycoprotein promoter (expressed in endothelial cells), fibroblast-specific protein 1 (FSP1) promoter (expressed in fibroblasts), fibronectin promoter (expressed in fibroblasts), fms-associated tyrosine kinase 1 (FLT1) promoter (expressed in endothelial cells), glial fibrillary acidic protein (GFAP) promoter (expressed in astrocytes), insulin promoter (expressed in pancreatic cells), integrin-a-2b (ITGA2B) promoter (megakaryocytes), intracellular adhesion molecule 2 (ICAM-2) promoter (endothelial cells), interferon-β (IFN-β) promoter (hematopoietic cells), keratin 5 promoter (expressed in keratinocytes), myoglobin (MB) promoter (expressed in muscle cells), myogenic differentiation 1 (MYOD1) promoter (expressed in muscle cells), nephropathy protein promoter (expressed in podocytes), bone γ-carboxyl glutamic acid protein 2 (OG-2) promoter (expressed in osteoblasts), 3-ketoacid CoA transferase 2B (Oxct2B) promoter (expressed in haploid spermatocytes), surface activating protein B (SP-B) promoter (lung cell), synapsin promoter (expressed in neuronal cells), or Wiskott-Aldrich syndrome protein (WASP) promoter (expressed in hematopoietic cells).

In some embodiments, the promoter is a non-cell-specific promoter. Exemplary non-cell-specific promoters include but are not limited to, cytomegalovirus (CMV) immediate-early promoter, viral simian virus 40 (SV40) (e.g., early or late) promoter, Moloney murine leukemia virus (MoMLV) LTR promoter, Rous sarcoma virus (RSV) LTR promoter, herpes simplex virus (HSV) (thymidine kinase) promoter, cowpox virus H5, P7.5 and Pll promoters, elongation factor 1-alpha (EF1a) promoter, early growth response 1 (EGR1) promoter, ferritin H (FerH) promoter, ferritin L (FerL) promoter, glyceraldehyde-3-phosphate dehydrogenase (GAPDH) promoter, eukaryotic translation initiation factor 4A1 (EIF4A1) promoter, heat shock 70 kDa protein 5 (HSPA5) promoter, heat shock protein 90 kDa-β member 1 (HSP90B1) promoter, heat shock protein 70 kDa (HSP70) promoter, β-kinesin (β-KIN) promoter, human R0SA26 locus (Irions et al., (2007) Nature Biotechnology 25, 1477-1482), ubiquitin C promoter (UBC), phosphoglycerate kinase-1 (PGK) promoter, cytomegalovirus enhancer/chicken β-actin (CAG) promoter, or β-actin promoter. The non-cell-specific promoters enable better versatility and expression efficiency of the described gene expression cassettes.

In some embodiments, the promoter is CMV, EF1a, SFH, CAG promoter, CBh, UBC, SFFV, SV40, RSV, mCMV, GAPDH, PGK, CASI, SMVP, GUSB (hGBp) or UCOE.

In some embodiments, the gene of interest is inserted downstream of the repressible operon. The repressible operon exerts a negative effect on viral packaging in the case where the gene of interest is expressed.

In the present disclosure, “a negative effect on viral packaging” refers to reducing the packaging efficiency of virus, slowing or hindering the release of virus, or directly killing the cells for packaging. Moreover, the gene of interest would take up excessive enzymes and resources related to RNA transcription and protein translation in cells if it is possible to be transcribed and translated in large quantities, thereby indirectly inhibiting the transcription and translation of the functional genes of the recombinant virus. The effects in these two aspects reduce the production efficiency of recombinant virus in the cells for packaging, result in an insufficient virus titer.

In some embodiments, the gene of interest has cytotoxicity to cells for packaging.

In some embodiments, the gene of interest is selected from a suicide gene, an apoptotic gene (or programmed cell death gene), or an oncogene.

Common suicide gene systems may include tk-GCV system, CD-5-FC system, gpt-6-TX system, P450 2BI-CPA system, or the like.

In the present disclosure, the “apoptotic gene” may be used interchangeably with “programmed cell death gene”, including but not limited to, Bcl-2 gene, P53 gene, cytochrome C gene, apoptotic protease activating factor 1 (Apaf-1) gene, genes of Caspase family protein, and the like.

Caspase family proteins may be categorized as initiator caspases (such as Caspase 8, Caspase 9, or Caspase 10) and effector caspases (such as Caspase 3, Caspase 6, or Caspase 7).

The main types of oncogenes include tyrosine kinases (such as src), other protein kinases (such as raf), G proteins (such as ras), growth factors (such as Sis), growth factor receptors (such as ErbB), and proteins within nucleus (such as transcription factor MYC).

In some embodiments, the gene of interest is a cell cycle-associated gene. For example, the gene of interest is a gene of Caspase family protein and a MAPK signaling pathway-related gene.

In some embodiments, the gene of interest directly inhibits replication and/or

assembly of virus.

For example, the product expressed by the gene of interest is a substance that is capable of inhibiting the elements related to replication and/or assembly of virus by directly cutting, interfering with, knocking out, or binding to the elements such as E2A, E4 VA RNA gene, rep and cap gene products, and the like. For example, The product is miRNA, siRNA, certain specific proteins, enzymes, and the like.

In some embodiments, the gene of interest is selected from GPR78 gene, Cdkn1a gene, and a gene from APOBEC family. The enzyme family of APOBEC, short for apolipoprotein B mRNA-editing enzyme catalytic polypeptide-like proteins, is a class of proteases found to be capable of deamination in recent years. The APOBEC enzyme family includes a family of activation-induced cytosine deaminase (AID), APOBEC1, APOBEC2, APOBEC3A-H, and APOBEC4. It was found that APOBEC has an inhibitory effect on adeno-associated virus.

In some embodiments, the gene of interest is selected from iCre gene and Caspase 8 gene.

In some embodiments, the vector for viral packaging further includes at least one of a reporter gene, an enhancer, an internal ribosome entry site, a terminator, or a polyadenylation signal.

According to yet another aspect, the present disclosure also relates to a viral vector packaging system, including the vector for viral packaging as described above and a backbone vector.

In some embodiments, the viral vector packaging system is an adeno-associated virus vector packaging system. In one of the embodiments, the backbone vectors are vectors carrying AAV rep and cap genes. The AAV rep and AAV cap genes can be present on the same vector or separated vectors. Alternatively, the AAV rep and AAV cap genes can be present on the above-mentioned vector for viral packaging. In one of the embodiments, the adeno-associated virus vector packaging system is an adeno-associated virus vector packaging system derived from any one of AAV1 to AAV13. In one of the embodiments, the adeno-associated virus vector packaging system further includes a viral helper vector. In one of the embodiments, the gene of repressor is packaged in the viral helper vector. In one of the embodiments, the viral helper vector is an adenovirus helper vector or a herpes virus helper vector.

In some embodiments, the viral vector packaging system is an adenovirus vector packaging system. The vector for viral packaging is used as a shuttle vector. In one of the embodiments, the adenovirus vector packaging system is based on the Adeasy system or the AdMAX system. The AdMax system is typically a two-plasmid system, consisting of a shuttle plasmid (such as pHBAd series, including overexpression, interference, and other types) and a backbone plasmid (such as pBHGlox(delta)E1, 3Cre). By using the Cre-loxP recombinase system, the AdMax system allows the production of a recombinant adenovirus with a higher titer from the adenovirus vector shuttle plasmid and the backbone plasmid (i.e., adenovirus genome plasmid) co-transfected into cells under the action of recombinase. The AdEasy packaging system is mainly characterized by using the Cre/loxP system in E. coli to complete the process of inserting the exogenous gene into adenovirus genome, thereby obtaining a circular recombinant adenovirus genome; and having the necessary elements for replication in bacteria. The recombinant adenovirus genome was digested, linearized, and then transfected into 293 cells, thereby obtaining a recombinant adenovirus without co-transfection with double plasmids.

According to yet another aspect, the present disclosure relates to a method for packaging a virus, including: transferring the viral vector packaging system as described above into a host cell, and performing packaging in the presence of a repressor.

In some embodiments, the virus is an adenovirus virus or an adeno-associated virus.

In some embodiments, the host cell is HEK-293 cell or a derivative thereof capable of providing E1 region of adenovirus.

In some embodiments, the derivative is at least one selected from a group consisting of HEK-293A, HEK-293S, HEK-293SG, HEK-293SGGD, HEK-293T, HEK-293T/17 SF, HEK-293H, HEK-293E, HEK-293-6E, HEK-293F, HEK-293 FT, HEK-293FTM, AAV-293 and GP2-293.

Embodiments of the present disclosure will be described in detail below in combination with examples.

### Example 1 Selection of Repressible Operon

A total of four transcriptional regulatory systems (i.e., CymR-CuO, tryptophan operon, diphtheria toxin repressor regulatory system, and lactose operon) were tested in the present disclosure.

At first, cis-acting elements of these four transcriptional regulatory systems were each introduced into lentiviral vectors to test whether the expression of exogenous gene was inhibited in a eukaryotic system. The lentiviral vector is a shuttle vector, which can be used as a general eukaryotic vector when transfecting cells alone. The conclusion is not limited to the lentivirus.

### I. Transfection of Plasmid

The method for transfecting plasmids into 293T cells specifically includes the follows.
1. One day before transfection, 293T cells were seeded onto a culture dish. One hour before transfection, the culture dish was taken out and, with the spent cell culture medium discarded and replaced with Opti-MEM medium, placed back in the incubator. Then, a complex of transfection reagent and plasmid was prepared in the following steps.
2. One or more plasmids prepared for transfection in equal proportions as needed were dissolved in Opti-MEM medium, gently mixed, and left standing to obtain a plasmid dilution solution.
3. The transfection reagent was dissolved in Opti-MEM medium, gently mixed and left standing to obtain a diluted solution of transfection reagent. The diluted solution of transfection reagent was added dropwise to the diluted solution of plasmid with gentle mixing during the addition, and then kept at room temperature for 15 to 25 mins, allowing the DNA and the transfection reagent to be thoroughly combined to form a stable transfection complex. The cell culture dish was taken out and, with the prepared complex of DNA and transfection reagent added, placed back in the incubator.
4. After 5 to 8 hours, the medium was aspirated and, after washing the dish with phosphate buffered saline (PBS), replaced with fresh complete medium for culturing. For the tryptophan operon group, the complete medium containing 0.3 mM tryptophan was used in the replacement. For the diphtheria toxin repressor regulatory system, divalent iron ions were needed.

### II. Validation of Different Repressible Operons

### 1. CymR-CuO System

The CymR-CuO system is a class of regulatable expression system that function by following principle.

CymR protein can specifically bind to CuO element in the absence of Cumate, thereby inhibiting the gene transcription. In the presence of Cumate, CymR bound to Cumate would detach from the CuO element, thus allowing normal expression of gene.

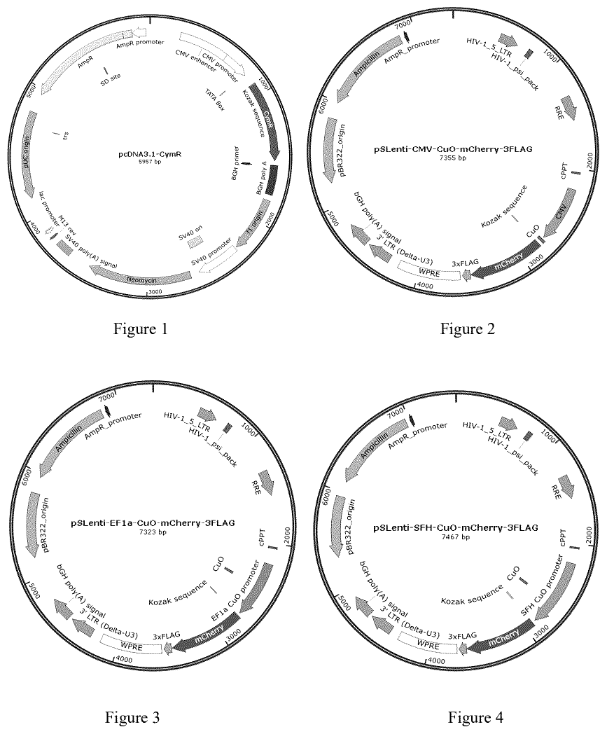

In the present disclosure, a series of promoter vectors CMV-CuO, EF1a-CuO, SFH-CuO and CAG-CuO were constructed. It was confirmed that the CuO element in cooperation with the CymR vector (pcDNA3.1-CymR) can effectively inhibit the gene expression. The plasmid map of pcDNA3.1-CymR is shown in FIG. 1. The plasmid maps of CMV-CuO, EF1a-CuO, SFH-CuO, and CAG-CuO promoter vectors are shown in FIGS. 2 to 5, respectively.

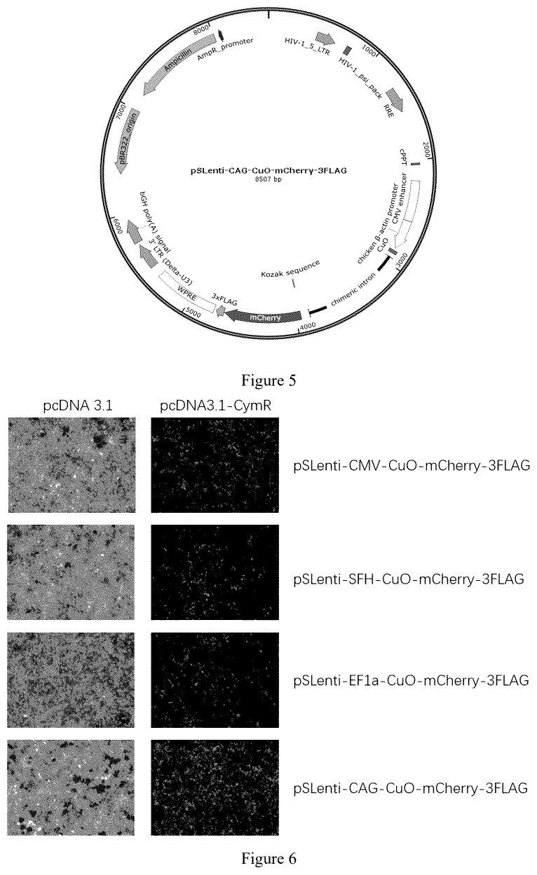

293T cells were co-transfected with the lentiviral vector encoding the CuO element and either pcDNA3.1 empty vector or pcDNA3.1-CymR. Fluorescence photographs were taken after 24 hours. It can be seen that the expression efficiency of CMV-CuO, EF1a-CuO, SFH-CuO and CAG-CuO promoters were significantly inhibited under the action of CymR protein (see FIG. 6).

### 2. Tryptophan Operon System

In the presence of tryptophan, the Trp repressor (TrpR) protein can specifically bind to the TrpO element and thus inhibits the gene transcription. This tryptophan operon system was found in prokaryotic cells and had long been used as an explanatory demonstration of gene regulation. However, this tryptophan operon system has not been applied in eukaryotic cells because tryptophan is necessary for the culture of mammalian cells.

In the present disclosure, a series of promoter vectors such as CMV-TrpO, EF1a-TrpO, SFH-TrpO, CAG-TrpO were constructed. It was confirmed that the TrpO element in cooperation with the TrpR vector (pcDNA3.1-TrpR) can effectively inhibit the gene expression in the presence of tryptophan. The plasmid map of CMV-TrpO is shown in FIG. 7. The design of plasmid maps of EF1a-TrpO, SFH-TrpO, and CAG-TrpO promoter vectors can be referred to FIGS. 2 to 5 on the basis of FIG. 7, which will not be repeated.

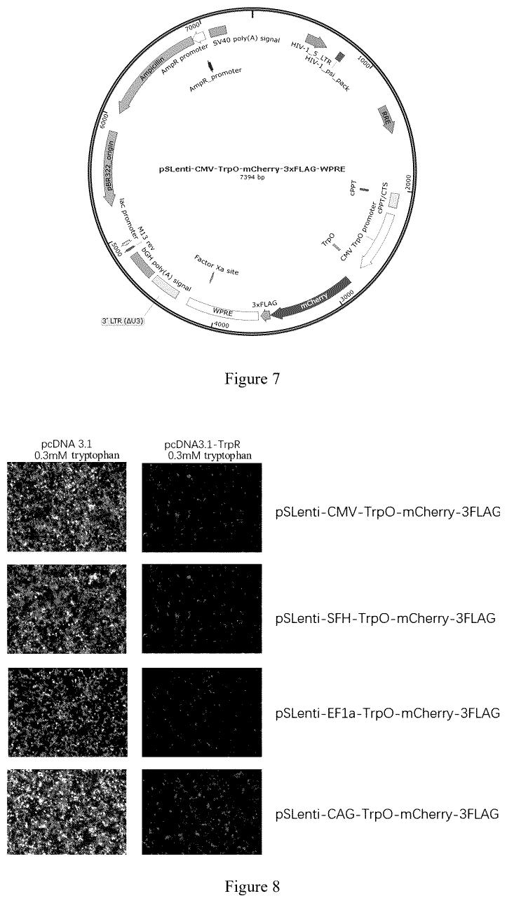

293T cells were co-transfected with the lentiviral vector encoding the TrpO element and either pcDNA3.1 empty vector or pcDNA3.1-TrpR, with 0.3 mM tryptophan added. Fluorescence photographs were taken after 24 hours. It can be seen that the expression efficiency of CMV-TrpO, EF1a-TrpO, SFH-TrpO and CAG-TrpO promoters were significantly inhibited under the action of TrpR protein (see FIG. 8).

### 3. Diphtheria Toxin Repressor Regulatory System

The diphtheria toxin repressor DtxR protein can specifically bind to the ToxO element and thus inhibits the gene transcription. In the present disclosure, a series of promoter vectors such as CMV-ToxO, EF1a-ToxO, SFH-TOXO, CAG-ToxO were constructed. 293T cells were co-transfected with the lentiviral vector encoding the ToxO element and either pcDNA3.1 empty vector or pcDNA3.1-DtxR, with divalent iron ions added. As a result, the gene of interest was not found to be significantly inhibited (see FIG. 10).

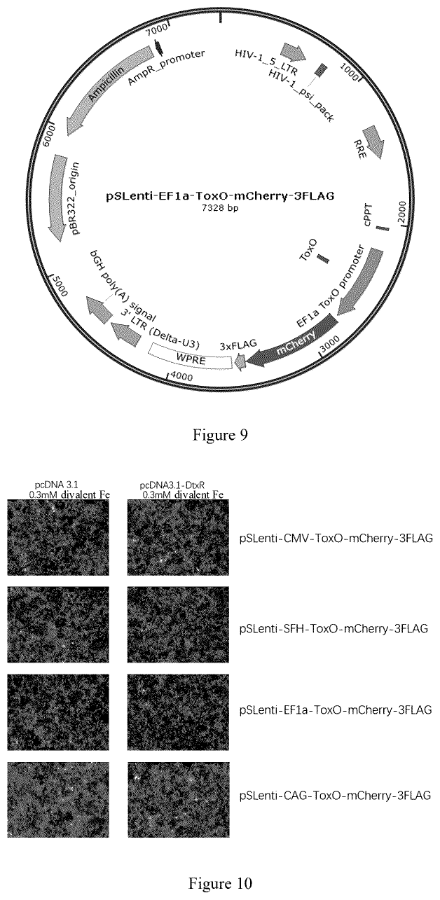

The plasmid map of EF1a-ToxO is shown in FIG. 9. The design of plasmid maps of CMV-ToxO, SFH-ToxO and CAG-ToxO promoter vectors can be referred to FIGS. 2 to 5 on the basis of FIG. 9, which will not be repeated.

### 4. Lactose Operon System

The lactose operon repressor LacI protein can specifically bind to the LacO element and thus inhibits the gene transcription. In the presence of isopropyl β-D-1-thiogalactopyranoside (IPTG), the LacI protein binds to the IPTG and is subjected to conformational change, thereby losing its binding ability and allowing the expression of downstream gene. The lactose operon system is widely used in prokaryotic cells for inducing the expression of exogenous genes.

In the present disclosure, a series of promoter vectors such as CMV-LacO, EF1a-LacO, SFH-LacO, CAG-LacO were constructed. 293T cells were co-transfected with the lentiviral vector encoding the LacO element and either pcDNA3.1 empty vector or pcDNA3.1-LacI. As a result, the gene of interest was not found to be significantly inhibited (see FIG. 12).

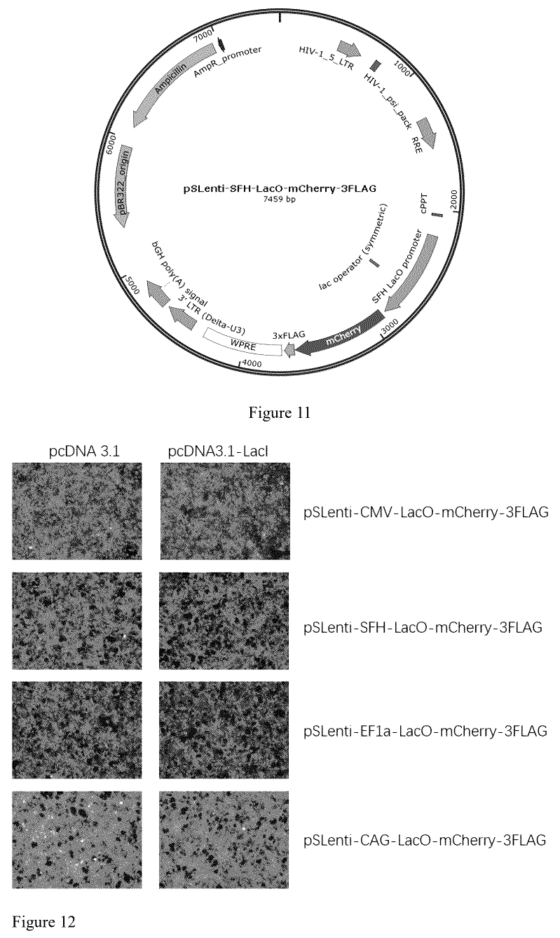

The plasmid map of SFH-LacO is shown in FIG. 11. The design of plasmid maps of CMV-LacO, EF1a-LacO, and CAG-LacO promoter vectors can be referred to FIGS. 2 to 5 on the basis of FIG. 11, which will not be repeated.

### III. Optimization of Insertion Sites of Repressible Operon Elements

### 1. TrpO Element

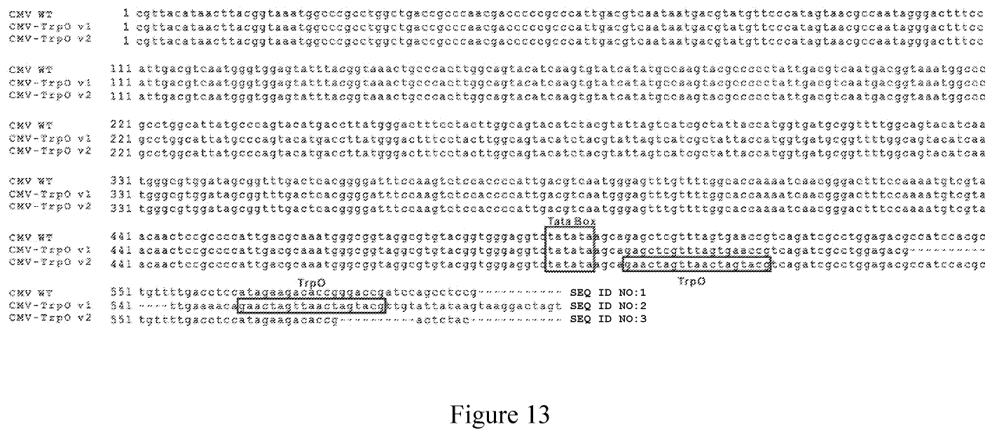

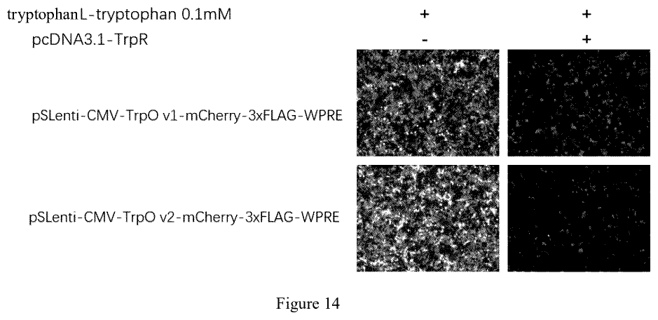

The distance between the TrpO element and the TATA Box of CMV can vary. The inventors have designed two promoters as shown in FIG. 13. At 48 hours after co-transfection with the plasmid encoding the TrpO element and pcDNA3.1-TrpR (with 0.1 mM tryptophan added), the fluorescence was observed. The experiment shows that varying the distance between the TrpO element and the TATA Box can result in different inhibitory effects (see FIG. 14). The TrpO v2 promoter sequence with a better inhibition effect was used in subsequent steps in the present disclosure.

### 2. CuO Element

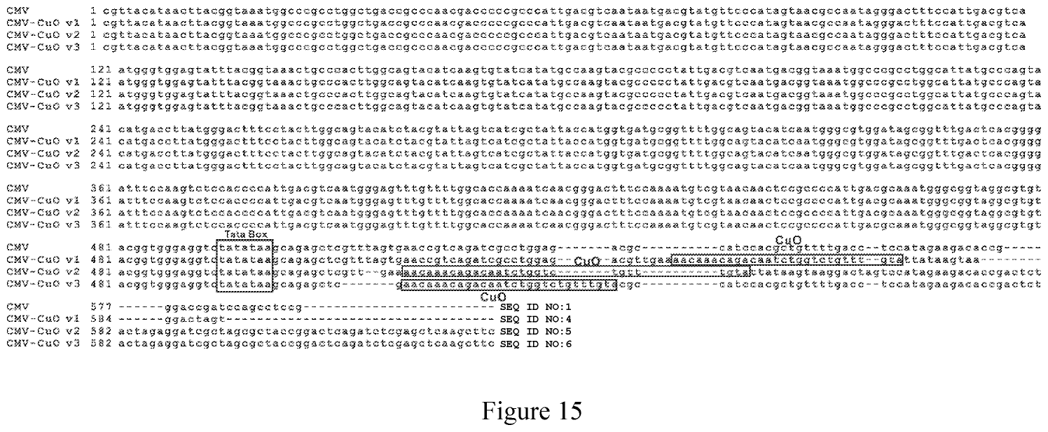

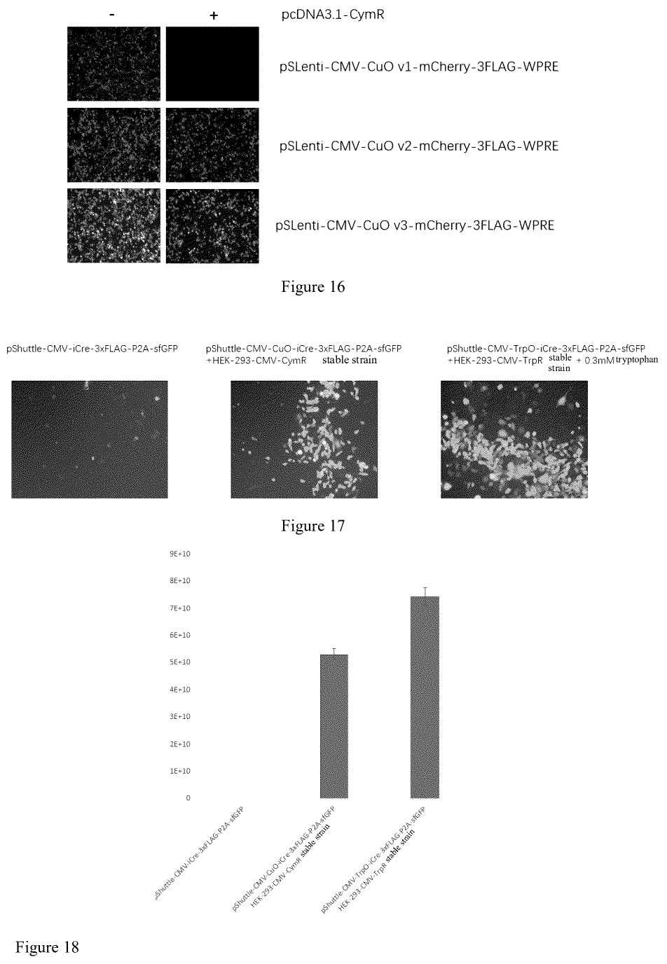

The distance between the CuO element and the TATA Box of CMV can vary. The inventors have designed three promoters, as shown in FIG. 15. At 48 hours after co-transfection with the plasmid encoding the CuO element and pcDNA3.1-CymR, the fluorescence was observed. The experiment shows that varying the distance between the CuO element and the TATA Box can result in different inhibitory effects (see FIG. 16). The CuO v1 promoter sequence with a better inhibition effect was used in subsequent steps in the present disclosure.

### Example 2 Packaging of Adenovirus and Expression of Gene of Interest

### I. Packaging of Adenovirus and Virus Titer Test

### 1. Construction of HEK-293-CMV-CymR and HEK-293-CMV-TrpR Stable Cell Lines

In this example, HEK-293-CMV-CymR and HEK-293-CMV-TrpR stable cell lines were constructed by the procedure specifically including the follows.

pcDNA3.1-CymR or pcDNA3.1-TrpR was linearized by using MfeI and BstBI (NEB Company) for double enzyme digestion. After 4 to 6 hours of enzyme digestion, the gel bands of linearized plasmids were recovered by using a gel extraction kit (QIAGEN). After the extraction was completed, the linearized plasmids were measured for concentration and confirmed by agarose gel electrophoresis.

One day before plasmid transfection, HEK 293 cells in good condition were selected, detached and then collected for cell counting, and the cells were seeded on a 24-well plate at 2×10⁵ cells per well with the plate shaken for thorough mixing, and cultured in an incubator containing 5% CO₂ at 37° C. The medium was replaced before transfection and the specific procedure is as follows. One hour before plasmid transfection, the 24-well plate with cells seeded the day before was taken out and, with the spent medium discarded and replaced with 0.5 mL of preheated Opti-MEM medium per well, placed back in the incubator containing 5% CO₂ for later transfection. The linearized plasmid (pcDNA3.1-CymR or pcDNA3.1-TrpR) prepared for transfection was dissolved in Opti-MEM medium, mixed gently, and left standing. The transfection reagent and Opti-MEM medium were mixed gently and left standing. The solution of transfection reagent mixture was added dropwise to the solution of plasmid mixture with gentle mixing during the addition, and then kept at room temperature for 15 to 25 minutes. The plate was taken out and, with the prepared complex of DNA and transfection reagent added without blowing up the cells, placed back in the incubator.

At 48 hours after transfection, G418 was added at 400 μg/ml for screening. The polyclonal cell populations were confirmed to express CymR or TrpR. HEK-293 monoclonal cells stably expressing pcDNA3.1-CymR or pcDNA3.1-TrpR were picked from the polyclonal cell populations by the limiting dilution method.

### 2. Packaging of Adenovirus

In this example, a method for packaging an adenovirus by using adenovirus AdEasy system is described. The method specifically includes the follows.

### (1) Recombination of Shuttle Plasmid

The shuttle plasmid carrying the exogenous gene (NLS-iCre or AceD81S) was recombined with the plasmid (pAdEasy) carrying most part of the adenovirus genome. The recombinant adenovirus genome plasmid was obtained through resistance screening. For the exogenous gene NLS-iCre, the shuttle plasmid was pShuttle-CMV-iCre-3×Flag-P2A-sfGFP, pShuttle-CMV-CuO-iCre-3×Flag-P2A-sfGFP, or pShuttle-CMV-TrpO-iCre-3×Flag-P2A-sfGFP. For the exogenous gene AceD8IS, the shuttle plasmid was pShuttle-CMV-AceD81S-mOrange2, pShuttle-CMV-TrpO-AceD81S-mOrange2, or pShuttle-CMV-CuO-AceD81S-mOrange2. Specific steps were as follows.
a. The shuttle plasmids (pShuttle-CMV-iCre-3×Flag-P2A-sfGFP, pShuttle-CMV-CuO-iCre-3×Flag-P2A-sfGFP, or pShuttle-CMV-TrpO-iCre-3×Flag-P2A-sfGFP; pShuttle-CMV-AceD81S-mOrange2, pShuttle-CMV-TrpO-AceD81S-mOrange2, or pShuttle-CMV-CuO-AceD81S-mOrange2) were linearized by enzyme digestion with Pmel (NEB).
b. After 4 to 6 hours of enzyme digestion, the gel bands of linearized plasmids were recovered by using a gel extraction kit (QIAGEN). After the extraction was completed, the linearized plasmids were measured for concentration and confirmed by agarose gel electrophoresis.
c. BJ5183 competent cells were taken out from −80° C., placed on ice quickly, and kept for 5 minutes to thaw the bacteria. The plasmid of interest (i.e., the linearized shuttle plasmid obtained after the treatments in a. and b.) and the pAdEasy plasmid were added to BJ5183 competent cells with gentle mixing, left standing on ice for 25 to 30 minutes, followed by heat shock in a 42° C. water bath for 90 seconds, and immediately transferred on ice for 2 minutes. After adding 800 μl sterile medium without antibiotics to the centrifuge tube, the cells were cultured with shaking at 37° C. at 200 rpm for 60 minutes, and centrifuged at 3000 rpm for 1 minute to harvest the bacteria cells with 100 μl supernatant left for resuspending. The bacterial suspension was spread evenly on the screening plate with kanamycin. The plate was incubated in a normal position for 10 minutes. After the liquid in the suspension was completely absorbed into the medium, the plate was placed in an inverted position and cultured in the incubator at 37° C. for 12 to 16 hours.
d. 10 to 20 monoclonal colonies were picked for plasmids mini-preparation and identified by Pac I enzyme digestion. The clones confirmed for positive recombination contained recombinant plasmids of interest.
e. The recombinant plasmids were linearized according to a. and b. for later transfection.

### (2) Transfection of Recombinant Plasmids

a. Seeding: one day before plasmid transfection, HEK 293 cells or HEK 293 cells stably expressing pcDNA3.1-CymR or pcDNA3.1-TrpR in good condition were selected, detached and then collected for cell counting, and the cells were seeded on a 6-well plate at 1×10⁶ cells per well with the plate shaken for thorough mixing, and cultured in an incubator containing 5% CO₂ at 37° C.
b. Replacement of medium before transfection: one hour before plasmid transfection, the 6-well plate with cells seeded the day before was taken out and, with the spent medium discarded and replaced with 1.5 mL of preheated Opti-MEM medium per well, placed back in the incubator containing 5% CO₂ for later transfection.
c. Preparation of transfection system for each well of 6-well plate

The linearized virus shuttle plasmid prepared for transfection was dissolved in Opti-MEM medium, mixed gently, and left standing. The transfection reagent and Opti-MEM medium were mixed gently and left standing. The solution of transfection reagent mixture was added dropwise to the solution of plasmid mixture with gentle mixing during the addition, and then kept at room temperature for 15 to 25 minutes.
d. The plate was taken out and, with the prepared complex of DNA and transfection reagent added without blowing up the cells, placed back in the incubator.

### (3) Amplification of Virus

The culture medium for the transfected cells was replaced every 3 to 5 days and observed for the release of the virus. After the release of the virus was observed, the virus was amplificated. For the tryptophan operon group (i.e., pShuttle-CMV-TrpO-iCre-3×Flag-P2A-sfGFP, or pShuttle-CMV-TrpO-AceD81S-mOrange2), the complete medium containing 0.3 mM tryptophan was used in the replacement.

### (4) Virus Harvest and Purification

A 50 ml centrifuge tube was ready for use. The cell supernatant was pipetted up and down using a 1 ml pipette to completely detach the cells in dishes. The cells and supernatant were both harvested into the 50 ml centrifuge tube, and subjected to repeated cycles of freezing (liquid nitrogen) and thawing, followed by centrifugation at 4000 rpm for 10 minutes. The supernatant was collected, filtered over a 0.22 μm filter, aliquoted into tubes for the virus, and stored at −80° C.

### 3. Adenovirus Titer Test

Adenovirus titer test kit was used for adenovirus detection based on the principle that adenovirus can infect and replicate in HEK293 cells and express a special capsid protein Hexon, which can be detected by immunostaining. The positive cells which were infected with the virus can be stained brown and counted so as to calculate the adenovirus titer with a formula. Specifically, the method includes the follows.
a. HEK293 cells in good condition were selected, seeded, and cultured at 37° C., 5% CO₂ for 1 hour.
b. Virus samples in 10-fold serial dilutions were prepared. 100 μl of the virus sample was added dropwise to the cells in each well with thorough mixing, and the plate was placed in an incubator containing 5% CO₂ at 37° C. for infection for 2 days.
c. The culture medium was gently removed. Pre-cooled methanol was added slowly along the side wall of wells of the 24-well plate for fixation at −20° C. for 20 minutes.
d. The cells were washed gently with the phosphate buffer saline (PBS) three times, 5 minutes each.
e. The plate was blocked with 1% bovine serum albumin (BSA) in PBS at 37° C. for 1 hour.
f. A solution of anti-Hexon antibody was added to each well before incubation at 37° C. for 1 hour.
g. The cells were washed gently with PBS three times, 5 minutes each.
h. Horseradish peroxidase-labeled secondary antibody was added to each well before incubation at 37° C. for 1 hour.
i. The cells were washed gently with PBS three times, 5 minutes each.
j. 1×DAB working solution was added to each well before incubation at a room temperature for 5 to 10 minutes.
k. After discarding diaminobenzidine (DAB), the cells were washed with PBS twice with 1 ml of PBS added to each well.
l. 5 fields of view were randomly selected for each well. The positive cells were counted under an optical microscope with a 10× objective lens.
m. The average number of positive cells per well was calculated to determine the adenovirus titer.

### II. Adenovirus Vectors Expressing NLS-iCre Gene

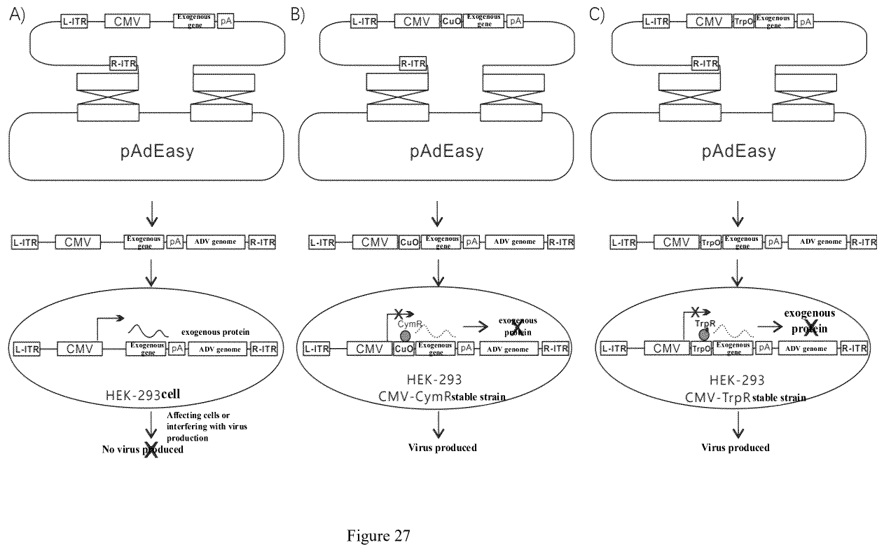

In the production of viruses, it was found that virus particles could not be effectively released and amplified in the case where the NLS-iCre gene was incorporated into a common adenovirus vector. This can be effectively solved by using stable cell lines in which the expression of the protein was inhibited in combination with CMV-CuO or CMV-TrpO promoter (see FIGS. 17 and 18). For the schematic diagram of the principle, see FIG. 27.

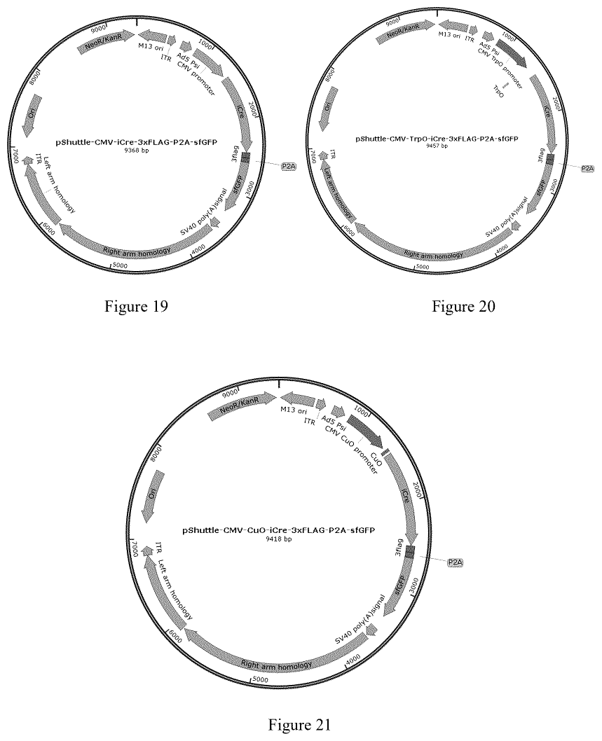

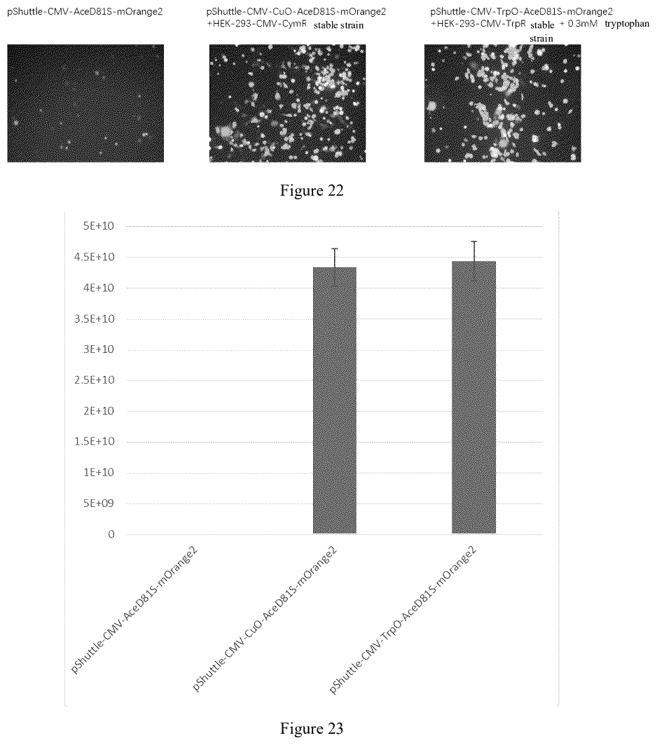

The plasmid maps of plasmids pShuttle-CMV-iCre-3×Flag-P2A-sfGFP, pShuttle-CMV-TrpO-iCre-3×Flag-P2A-sfGFP, and pShuttle-CMV-CuO-iCre-3×Flag-P2A-sfGFP used in this example are shown in FIGS. 19, 20, and 21, respectively.

III. Adenovirus Vectors expressing AceD81S Gene

In the production of viruses, it was found that virus particles could not be effectively released and amplified in the case where the AceD81S gene was incorporated into a common adenovirus vector. This can be effectively solved by using stable cell lines in which the expression of the protein was inhibited in combination with CMV-CuO or CMV-TrpO promoter (see FIGS. 17 and 18). For the schematic diagram of the principle, see FIG. 27.

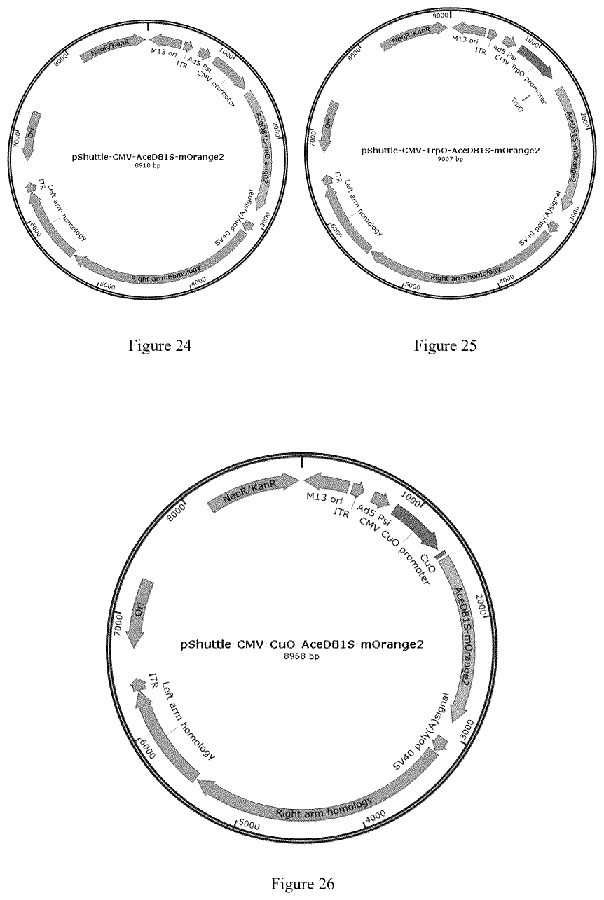

The plasmid maps of plasmids pShuttle-CMV-AceD81S-mOrange2, pShuttle-CMV-TrpO-AceD81S-mOrange2, and pShuttle-CMV-CuO-AceD81S-mOrange2 used in this example are shown in FIGS. 24, 25, and 26, respectively.

### Example 3 Packaging of Adeno-Associated Virus and Expression of Gene of Interest

### I. Packaging of Adeno-Associated Virus and Virus Titer Test

### 1. Packaging of Adeno-Associated Virus

A method for virus packaging by using an adeno-associated virus vector (AAV) specifically includes the follows.
(1) One day before transfection, 293T cells were seeded onto a culture dish. One hour before transfection, the culture dish was taken out and, with the spent cell culture medium discarded and replaced with Opti-MEM medium, placed back in the incubator. Then, a complex of transfection reagent and plasmid was prepared in the following steps.
(2) The viral vector plasmids prepared for transfection (including a serotype plasmid, a helper plasmid and a shuttle plasmid) were dissolved in Opti-MEM medium, gently mixed, and left standing to obtain a plasmid dilution solution. The serotype plasmid was pAAV9, pAAV9-CMV-CymR, or pAAV9-CMV-TrpR. The helper plasmid was pHelper, pHelper-CMV-CymR, or pHelper-CMV-TrpR. For the gene of interest GPR78, the shuttle plasmid was pAAV-CMV-GPR78-3×FLAG-WPRE, pAAV-CMV-CuO-GPR78-3×FLAG-WPRE, or pAAV-CMV-TrpO-GPR78-3×FLAG-WPRE. For the gene of interest Cdkn1a, the shuttle plasmid was pAAV-CMV-Cdkn1a-3×FLAG-WPRE, pAAV-CMV-CuO-Cdkn1a-3×FLAG-WPRE, or pAAV-CMV-TrpO-Cdkn1a-3×FLAG-WPRE.
(3) The transfection reagent was dissolved in Opti-MEM medium, gently mixed and left standing to obtain a diluted solution of transfection reagent. The diluted solution of transfection reagent was added dropwise to the diluted solution of plasmid with gentle mixing during the addition, and then kept at room temperature for 15 to 25 mins, allowing the DNA and the transfection reagent to be thoroughly combined to form a stable transfection complex. The cell culture dish was taken out and, with the prepared complex of DNA and transfection reagent added, placed back in the incubator.
(4) After 5 to 8 hours, the medium was aspirated and, after washing the dish with phosphate buffered saline (PBS), replaced with fresh complete medium for culturing. For the tryptophan operon group (i.e., pAAV-CMV-TrpO-GPR78-3×FLAG-WPRE, or pAAV-CMV-TrpO-Cdkn1a-3×FLAG-WPRE), the complete medium containing 0.3 mM tryptophan was used in the replacement.
(5) 60 to 72 hours after transfection, the cells were completely detached from the culture dish by repeated pipetting onto the culture dish with a pipette tip. The cell sample and supernatant were both collected.
(6) The collected cell sample was repeatedly frozen at −80° C. and thawed at 37° C. After centrifugation, the supernatant was collected, and filtered over a 0.45 μm PVDF filter to remove cell debris. Then the collected adeno-associated viruses were purified by using the AAV purification kit, thereby obtaining purified adeno-associated viruses, which were stored in a refrigerator at −80° C.

### 2. Titer Test of Adeno-Associated Virus

### (1) DNA Extraction of Adeno-Associated Virus

To 20 μl of concentrated virus stock, 1 μL of RNase-free DNase was added with thorough mixing. The mixture was incubated at 37° C. for 30 mins and centrifuged at 10000 rpm for 10 mins. 20 μL of the supernatant was collected and diluted in 80 μL of dilution buffer in a fresh sterile tube with thorough mixing. The mixture was incubated in a metal bath at 100° C. for 10 mins, and cooled naturally to room temperature. After adding 3 μL of proteinase K, the mixture was further incubated at 37° C. for 60 mins, incubated in a metal bath at 100° C. for 10 mins, and then cooled to room temperature.

### (2) Titer Test by Real-Time PCR

Using the above sample that has been diluted as a template, the titer of adeno-associated virus was determined by Real-time PCR test method. The Real-time quantitative PCR was performed on ABI7500 instrument. Reagent SYBR Master Mixture used was from TAKARA.
a. The reaction system was prepared according to the following proportions:
SYBR premix ex taq: 10 μl;
ROX: 0.4 μl;
Forward primer (25 μM): 0.5 μl;
Reverse primer (25 μM): 0.5 μl;
Genomic DNA: 2.0 μl; and
water: 6.6 μl.
b. A procedure of two-step Real-time quantification was performed, including pre-denaturation at 95° C. for 15 seconds; and denaturation at 95° C. for 5 seconds and annealing extension at 60° C. for 34 seconds in each cycle, with a total of 40 cycles. The absorbance value was read during each extension stage.

The PCR procedure was follows:
Cycle 1: (1×)
Step 1:95.0° C. for 15 seconds;
Cycle 2: (40×)
Step 1:95.0° C. for 5 seconds,
Step 2:60.0° C. for 34 seconds; and
initiation of data collection and real-time analysis.
c. A melting curve was prepared. After PCR, denaturation was performed at 95° C. for 1 minute, followed by cooling down to 55° C. to allow fully binding of the DNA double strands. Then, the reaction temperature was increased from 55° C. to 95° C., with an increase of 0.5° C. per cycle and each step holding for 30 seconds, as the absorbance value was read.

The procedure for preparing a melting curve was as follows:
Cycle 3: (1×)
Step 1:95.0° C. for 1 minute;
Cycle 4: (1×)
Step 1:55.0° C. for 1 minute;
Cycle 5: (81×)
Step 1:55.0° C. to 95.0° C., each step holding for 30 seconds,
the set temperature was increased by 0.5° C. after every 2 cycles in Cycle 5.
   II. Adeno-Associated Virus Vector Expressing GPR78 (Glucose-Regulated Protein, 78 kDa) Gene

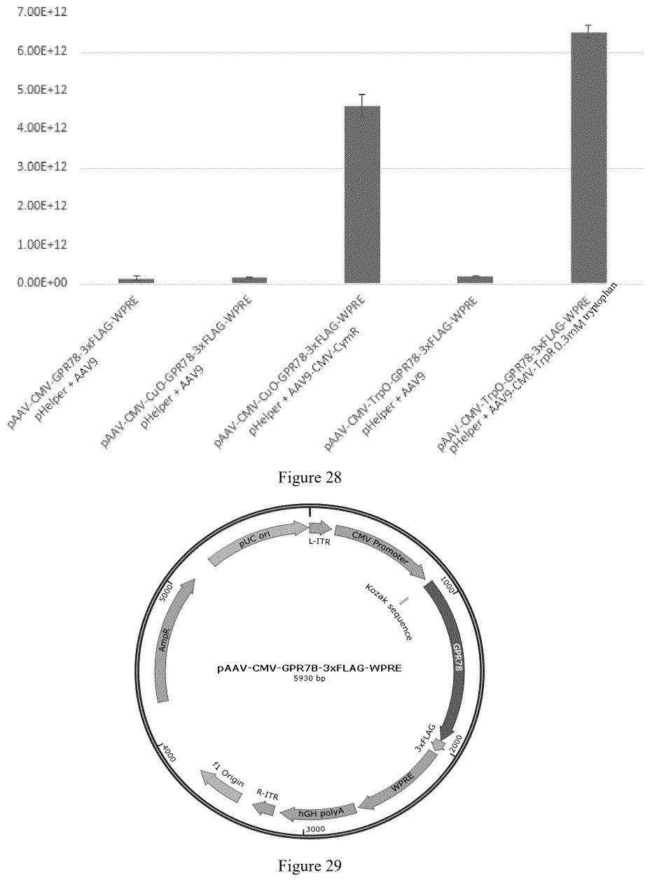

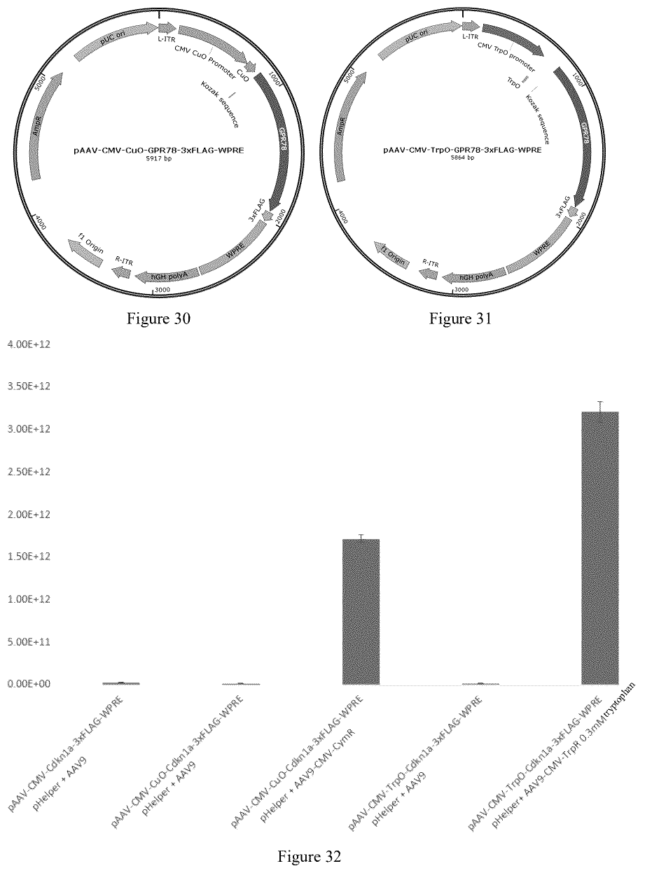

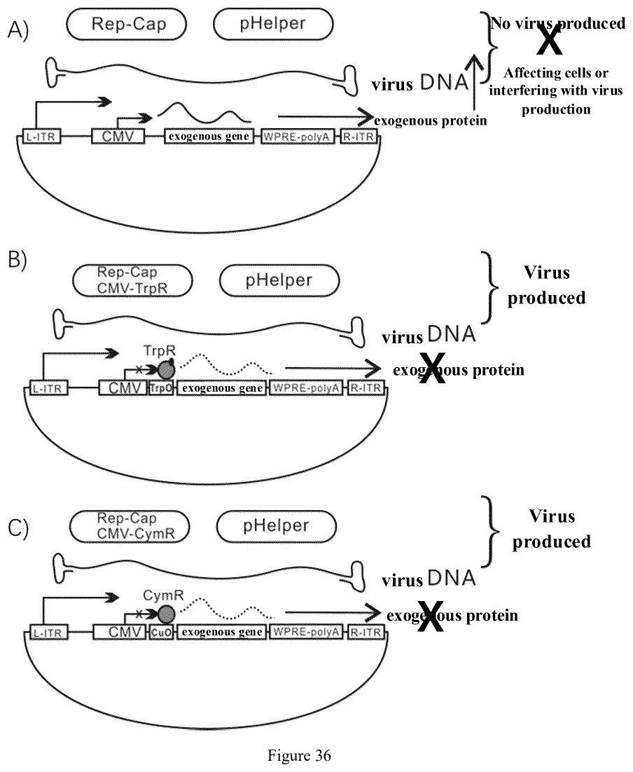

GRP78 is a type of molecular chaperone protein that assists newly generated protein molecules to fold into the correct three-dimensional configuration. In the production of viruses, it was found that virus particles could be hardly obtained in the case where the GPR78 gene was incorporated into a common adeno-associated virus vector, consistent with existing reports. The problem of low virus release can be effectively solved by inhibition of the expression of the protein in cooperation with CMV-CuO or CMV-TrpO promoter. For schematic diagram of the principle, see FIG. 36. The results of virus titer test are shown in FIG. 28. The plasmid maps of plasmids pAAV-CMV-GPR78-3×FLAG-WPRE, pAAV-CMV-CuO-GPR78-3×FLAG-WPRE, and pAAV-CMV-TrpO-GPR78-3×FLAG-WPRE used in this example are shown in FIGS. 29, 30, and 31, respectively.

Iii. Adeno-Associated Virus Vector Expressing Cdkn1a Gene

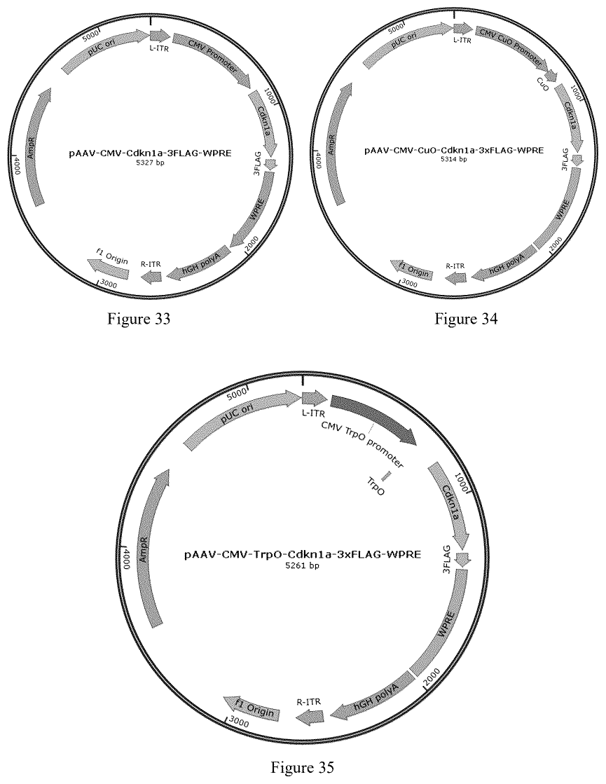

In the production of viruses, it was found that a very low total amount of viral particles could be obtained in the case where Cdkn1a gene was incorporated into a common adeno-associated virus vector. The problem of low virus release can be effectively solved by inhibition of the expression of the protein in cooperation with CMV-CuO or CMV-TrpO promoter. For schematic diagram of the principle, see FIG. 36. The results of virus titer test are shown in FIG. 32. The plasmid maps of plasmids pAAV-CMV-Cdkn1a-3×FLAG-WPRE, pAAV-CMV-CuO-Cdkn1a-3×FLAG-WPRE, and pAAV-CMV-TrpO-Cdkn1a-3×FLAG-WPRE used in this example are shown in FIGS. 33, 34, and 35, respectively.

The technical features of the examples described above can be combined arbitrarily. For the sake of conciseness, all possible combinations of the technical features of the above examples are not described. However, as long as these combinations of the technical features do not contradict each other, they should be considered to be within the scope of this specification.

The examples described above in a specific and detailed manner merely express several embodiments of the present disclosure. However, they should not be construed as a limitation to the scope of the present disclosure. It should be noted that a number of variations and improvements can be made for a person of ordinary skill in the art without departing from the conception of the present disclosure, all of which fall within the protection scope of the present disclosure. Therefore, the protection scope of the present disclosure shall be subject to the appended claims.

1. A vector for viral packaging, comprising two inverted terminal repeats, and a gene expression cassette inserted between the two inverted terminal repeats,
wherein the gene expression cassette comprises a promoter, a repressible operon and an optional gene of interest which are linked in sequence, and
the repressible operon is capable of repressing expression of the gene of interest downstream of the repressible operon in the presence of a repressor.

2. The vector for viral packaging according to claim 1, wherein the vector for viral packaging is a vector for adenovirus packaging or a vector for adeno-associated virus packaging.

3. The vector for viral packaging according to claim 2, wherein the repressible operon is selected from a tryptophan operon and/or a Cumate-CuO regulable system.

4. The vector for viral packaging according to claim 3, wherein a distance between a site where a TrpO element of a tryptophan operon is inserted into the gene expression cassette and a TATA BOX of the promoter is less than or equal to 18 nucleotides.

5. The vector for viral packaging according to claim 3, wherein a distance between a site where a CuO element of a Cumate-CuO regulable system is inserted into the gene expression cassette and a TATA BOX of the promoter is 40 to 50 nucleotides.

6. The vector for viral packaging according to claim 2, wherein the promoter is CMV, EF1a, SFH, CAG, CBh, UBC, SFFV, SV40, RSV, mCMV, GAPDH, PGK, CASI, SMVP, GUSB, or UCOE.

7. The vector for viral packaging according to claim 1, wherein a gene of interest is inserted downstream of the repressible operon, and
the repressible operon exerts a negative effect on viral packaging in the case where the gene of interest is expressed.

8. The vector for viral packaging according to claim 7, wherein the gene of interest has cytotoxicity to cells for packaging.

9. The vector for viral packaging according to claim 8, wherein the gene of interest is selected from a suicide gene, an apoptotic gene, or an oncogene.

10. The vector for viral packaging according to claim 7, wherein the gene of interest is a cell cycle-associated gene.

11. The vector for viral packaging according to claim 7, wherein the gene of interest directly inhibits replication and/or assembly of virus.

12. The vector for viral packaging according to claim 1, wherein the vector for viral packaging further comprises at least one of a reporter gene, an enhancer, an internal ribosome entry site, a terminator, or a polyadenylation signal.

13. A viral vector packaging system, comprising the vector for viral packaging according to claim 1 and a backbone vector.

14. A method for packaging a virus, comprising:
transferring a viral vector packaging system of claim 13 into a host cell, and
performing packaging in the presence of a repressor.

15. The method according to claim 14, wherein the host cell is HEK-293 cell or a derivative thereof capable of providing E1 region of adenovirus.

16. The method according to claim 15, wherein the derivative is at least one selected from a group consisting of HEK-293A, HEK-293S, HEK-293SG, HEK-293SGGD, HEK-293T, HEK-293T/17 SF, HEK-293H, HEK-293E, HEK-293-6E, HEK-293F, HEK-293 FT, HEK-293FTM, AAV-293 and GP2-293.


SEQUENCE LISTING (6 sequences)

SEQ ID NO: 1 - DNA, 594 bp, Artificial Sequence
  CMV WT
cgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccg

SEQ ID NO: 2 - DNA, 591 bp, Artificial Sequence
  CMV-TrpO v1
cgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgttgaaaacagaactagttaactagtacgttgtattataagtaaggactagt

SEQ ID NO: 3 - DNA, 583 bp, Artificial Sequence
  CMV-TrpO v2
cgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagaactagttaactagtacgtcagatcgcctggagacgccatccacgctgttttgacctccatagaagacaccgactctac

SEQ ID NO: 4 - DNA, 591 bp, Artificial Sequence
  CMV-CuO v1
cgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgttgaaaacaaacagacaatctggtctgtttgtattataagtaaggactagt

SEQ ID NO: 5 - DNA, 630 bp, Artificial Sequence
  CMV-CuO v2
cgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgttgaaaacaaacagacaatctggtctgtttgtattataagtaaggactagtccatagaagacaccgactctactagaggatcgctagcgctaccggactcagatctcgagctcaagcttc

SEQ ID NO: 6 - DNA, 630 bp, Artificial Sequence
  CMV-CuO v3
cgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgaacaaacagacaatctggtctgtttgtacgccatccacgctgttttgacctccatagaagacaccgactctactagaggatcgctagcgctaccggactcagatctcgagctcaagcttc